SEQUENCE LISTING (51 sequences)

SEQ ID NO: 1 - protein, 100 aa, Homo sapien
MQKPCKENEGKPKCSVPKREEKRPYGEFERQQTEGNFRQRLLQSLEEFKEDIDYRHFKDEEMTREGDEMERCLEEIRGLRKKFRALHSNHRHSRDRPYPI

SEQ ID NO: 2 - protein, 111 aa, Homo sapien
MANIHQENEEMEQPMQNGEEDRPLGGGEGHQPAGNRRGQARRLAPNFRWAIPNRQINDGMGGDGDDMEIFMEEMREIRRKLRELQLRNCLRILMGELSNHHDHHDEFCLMP

SEQ ID NO: 3 - protein, 13 aa, Homo sapien
RGLRKKFRALHSN

SEQ ID NO: 4 - protein, 13 aa, Homo sapien
REIRRKLRELQLR

SEQ ID NO: 5 - protein, 9 aa, Homo sapien
LALAGLDIN

SEQ ID NO: 6 - protein, 10 aa, Human immunodeficiency virus
LPPLERLTLD

SEQ ID NO: 7 - protein, 10 aa, Homo sapien
LSFDESLALC

SEQ ID NO: 8 - protein, 12 aa, Homo sapien
ALQKKLEELELD

SEQ ID NO: 9 - protein, 157 aa, Homo sapien
MDKPRKENEEEPQSRPRPMRRGLRWSTLPKSSPPRSSLRRSSPRRRSSFLRSSCLSSCLRCSSRRTPSAGLSRKDLFEVRPPMEQPPCGVGKHNLEEGIFKERLARSRPQFRGDIHGRNLSNEEMIQAADELEEMKRVRNKLMIMHWRAKRGGPYPI

SEQ ID NO: 10 - DNA, 23 bp, Artificial Sequence
  Primer
aagcagtggtaacaacgcagagt

SEQ ID NO: 11 - DNA, 21 bp, Artificial Sequence
  Primer
ctaatacgactcactatgggc

SEQ ID NO: 12 - DNA, 20 bp, Artificial Sequence
  Primer
accacagtccatgccatcac

SEQ ID NO: 13 - DNA, 21 bp, Artificial Sequence
  Primer
tccaccaccctgttgcttgta

SEQ ID NO: 14 - DNA, 22 bp, Artificial Sequence
  Primer
ggagatactggacaacccactg

SEQ ID NO: 15 - DNA, 20 bp, Artificial Sequence
  Primer
ccaactcccttccaaagtgc

SEQ ID NO: 16 - DNA, 20 bp, Artificial Sequence
  Primer
aatcgcaaggcacctctgag

SEQ ID NO: 17 - DNA, 20 bp, Artificial Sequence
  Primer
gatctggtttaccatctttt

SEQ ID NO: 18 - DNA, 19 bp, Artificial Sequence
  Primer
agctgctgtgccacgaggt

SEQ ID NO: 19 - DNA, 19 bp, Artificial Sequence
  Primer
actggcatcctcacaggtc

SEQ ID NO: 20 - DNA, 18 bp, Artificial Sequence
  Primer
taatgcacaaatggatac

SEQ ID NO: 21 - DNA, 18 bp, Artificial Sequence
  Primer
attgccataggaagaaag

SEQ ID NO: 22 - DNA, 21 bp, Artificial Sequence
  Primer
gacaccagctctccagcctgc

SEQ ID NO: 23 - DNA, 21 bp, Artificial Sequence
  Primer
ggaagggcttctggtctgggg

SEQ ID NO: 24 - DNA, 20 bp, Artificial Sequence
  Primer
ccactgagggttcccagcac

SEQ ID NO: 25 - DNA, 20 bp, Artificial Sequence
  Primer
ggaaacacgaaggggagggt

SEQ ID NO: 26 - DNA, 21 bp, Artificial Sequence
  Primer
cctggttgtgaaaatacatga

SEQ ID NO: 27 - DNA, 21 bp, Artificial Sequence
  Primer
tgacattaacaagattttgcc

SEQ ID NO: 28 - DNA, 21 bp, Artificial Sequence
  Primer
tggtcaccaggacaaagacac

SEQ ID NO: 29 - DNA, 20 bp, Artificial Sequence
  Primer
atcctgccagcaagctgaca

SEQ ID NO: 30 - DNA, 22 bp, Artificial Sequence
  Primer
cgcagcccgaagattcactatg

SEQ ID NO: 31 - DNA, 24 bp, Artificial Sequence
  Primer
cttattaccaaggacactctatct

SEQ ID NO: 32 - DNA, 23 bp, Artificial Sequence
  Primer
cggagaactgtgagaagctcgtc

SEQ ID NO: 33 - DNA, 20 bp, Artificial Sequence
  Primer
ggtggggctaggctagaagg

SEQ ID NO: 34 - DNA, 20 bp, Artificial Sequence
  Primer
gcaacatgccagtggcccgg

SEQ ID NO: 35 - DNA, 20 bp, Artificial Sequence
  Primer
atcctcctcctcgtcgtctc

SEQ ID NO: 36 - DNA, 20 bp, Artificial Sequence
  Primer
gattgaccacagtcttatct

SEQ ID NO: 37 - DNA, 23 bp, Artificial Sequence
  Primer
taagaggtctactcattgctcac

SEQ ID NO: 38 - DNA, 20 bp, Artificial Sequence
  Primer
gcacctggcccaactgtaac

SEQ ID NO: 39 - DNA, 19 bp, Artificial Sequence
  Primer
ccagctgctggctcacctt

SEQ ID NO: 40 - DNA, 22 bp, Artificial Sequence
  Primer
tgtgatttatagttgcgatttg

SEQ ID NO: 41 - DNA, 22 bp, Artificial Sequence
  Primer
tgtgatttatagttgcgatttg

SEQ ID NO: 42 - DNA, 22 bp, Artificial Sequence
  Primer
tgtgacttacagttgcgatgtc

SEQ ID NO: 43 - DNA, 22 bp, Artificial Sequence
  Primer
ccagccacctgtttctgctgct

SEQ ID NO: 44 - DNA, 21 bp, Artificial Sequence
  Primer
attaggagttgacgtgaatcg

SEQ ID NO: 45 - DNA, 23 bp, Artificial Sequence
  Primer
ctattccaatcattaattcaccc

SEQ ID NO: 46 - DNA, 20 bp, Artificial Sequence
  Primer
atcaggagctgacgtgaacg

SEQ ID NO: 47 - DNA, 24 bp, Artificial Sequence
  Primer
ctattccagtcattagattcaccc

SEQ ID NO: 48 - DNA, 24 bp, Artificial Sequence
  Primer
gtatgtgttggtgtgtggagaaag

SEQ ID NO: 49 - DNA, 24 bp, Artificial Sequence
  Primer
aaacctaaaccttacaaaaccgcg

SEQ ID NO: 50 - DNA, 24 bp, Artificial Sequence
  Primer
gtatgtgttggtgtgtggagaaag

SEQ ID NO: 51 - DNA, 25 bp, Artificial Sequence
  Primer
aggcctgggccttgcaaggcccgcg

(19) United States
(12) Patent Application Publication
(10) Pub. No.: US 2007/0004621 A1
(43) Pub. Date: Jan. 4, 2007

(54) Bex4 nucleic acids, polypeptides, and method of using

(76) Inventors: Viji Shridhar, Rochester, MN (US); Jeremy Chien, Rochester, MN (US)

Correspondence Address: Fish & Richardson, 60 South Sixth Street, Suite 3300, Minneapolis, MN, 55402, US

(21) Appl. No.: 10/543,859

(22) PCT Filed: Feb. 12, 2004
(86) PCT No.: PCT/US04/04413

§ 371 Date: May 30, 2006

Related U.S. Application Data
(60) Provisional application No. 60/446,877, filed on Feb. 12, 2003.

(51) Int. Cl. A61K 48/00; A61K 38/54; C12Q 1/68; G01N 33/574; C12N 15/09

(52) U.S. Cl. 514/12; 514/44; 435/6; 435/7.23; 435/320.1

(57) ABSTRACT

Bex4 nucleic acids and polypeptides are provided, as are methods of using the nucleic acids and polypeptides.

### TECHNICAL FIELD

This invention relates to Bex4 nucleic acids and proteins, and to methods for using the nucleic acids and proteins to treat ovarian cancer patients and to detect cancer recurrence in ovarian cancer patients.

### BACKGROUND

Each year in the United States, 27,000 women are diagnosed with ovarian cancer (OvCa), resulting in approximately 14,000 fatalities (Shridhar et al. (2001) Cancer Res. 61:5895-5904). Increased understanding of genetic alterations associated with cancer and the functional consequences of such alterations in cancer would provide groundwork for development of early detection markers, novel therapeutic targets, and better management of cancers such as OvCa.

### SUMMARY

The invention is based on the discovery that the gene encoding Bex4 (also known as the ProApoptotic Protein on chromosome X (PAPX)) is down regulated in cancer cells (e.g., OvCa cells and epithelial cancer cells). Bex4 may be useful for treating cancer patients, as Bex4 expression induces apoptosis and reduces colony formation efficiency. Furthermore, the methylation status of the Bex4 gene may be a useful indicator of an OvCa patient's prognosis, as cells expressing Bex4 (e.g., normal ovarian epithelial cells) display lower levels of DNA methylation than cells that do not express Bex4 (e.g., tumor cells).

In one aspect, the invention features a vector containing an isolated nucleic acid encoding a polypeptide that has the amino acid sequence set forth in SEQ ID NO: 1 or a fragment thereof. The invention also features a vector containing an isolated nucleic acid encoding a Bex4 polypeptide, where the amino acid sequence of the Bex4 polypeptide contains a variant relative to the amino acid sequence set forth in SEQ ID NO:1.

In another aspect, the invention features a method for killing a tumor cell. The method can include administering to the tumor cell a nucleic acid that encodes a Bex4 polypeptide. The Bex4 polypeptide can have the amino acid sequence set forth in SEQ ID NO:1 or a fragment thereof. The amino acid sequence of the Bex4 polypeptide can contain a variant relative to the amino acid sequence set forth in SEQ ID NO:1. A vector containing the nucleic acid can be administered to the tumor cell. The tumor cell can be selected from the group consisting of an ovarian tumor cell, a cervical tumor cell, a brain tumor cell, a breast tumor cell, a prostate tumor cell, and a hepatic tumor cell.

In another aspect, the invention features a method for killing a tumor cell. The method can include administering to the tumor cell a purified Bex4 polypeptide. The Bex4 polypeptide can have the amino acid sequence set forth in SEQ ID NO:1 or a fragment thereof. The amino acid sequence of the Bex4 polypeptide can contain a variant relative to the amino acid sequence set forth in SEQ ID NO:1. The tumor cell can be selected from the group consisting of an ovarian tumor cell, a cervical tumor cell, a brain tumor cell, a breast tumor cell, a prostate tumor cell, and a hepatic tumor cell.

In yet another aspect, the invention features a method for determining the predisposition of an individual to develop cancer. The method can include measuring the level of Bex4 polypeptide in a biological sample from the individual. The individual can be predisposed to develop ovarian cancer if the level of Bex4 polypeptide in the biological sample is lower than the level of Bex4 polypeptide in a biological sample from a normal individual. The cancer can be selected from the group consisting of ovarian cancer, breast cancer, prostate cancer, cervical cancer, brain cancer, and liver cancer.

The invention also features a method for detecting cancer recurrence in an individual diagnosed with and treated for ovarian cancer. The method can include measuring the level of Bex4 methylation in a biological sample from the individual. The presence of hypermethylation can indicate cancer recurrence, and the absence of hypermethylation can indicate that cancer has not recurred. The cancer can be selected from the group consisting of ovarian cancer, breast cancer, prostate cancer, cervical cancer, brain cancer, and liver cancer.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram depicting the structure of the gene encoding Bex4.

FIG. 2 is the amino acid sequence of Bex4 (SEQ ID NO:1).

FIG. 3A is a homology alignment of Bex4 (PAPX; SEQ ID NO:1) and the p75NTR-associated death executor (NADE; SEQ ID NO:2). The nuclear export signal of NADE is boxed. Identical amino acids (*), strongly similar amino acids (:), and weakly similar amino acids (.) are indicated under the alignment. FIG. 3B is an alignment of select segments of Bex4 (PAPX; SEQ ID NO:3), NADE, (SEQ ID NO:4), PKI (SEQ ID NO:5), HIV rev (SEQ ID NO:6), MDM2 (SEQ ID NO:7), and MAPKK (SEQ ID NO:8), and shows that Bex4 contains a conserved Rev-like NES motif. FIG. 3C is a homology alignment of Bex4 (PAPX; SEQ ID NO:1) and the transcription elongation factor A-like 1 (TCEAL1; SEQ ID NO:9). FIG. 3D shows the secondary structure prediction of Bex4, indicating the presence of a helix-turn-helix motif.

FIG. 4 is a diagram depicting the genomic structure of the Bex4 gene, showing the positions of SmaI sites 1, 2, 3A, 3B, and 4. White boxes indicate the positions of the three exons, and the hatched box shows the open reading frame.

FIG. 5A is a graph plotting the rates of apoptosis in cells transfected with a Bex4 expression vector or with empty vector, with the apoptosis rate indicated by level of 7-AAD labeling. FIG. 5B is a column graph showing the number of colonies formed by Bex4-expressing cells and vector-transfected cells.

### DETAILED DESCRIPTION

In general, the invention provides materials and methods related to killing a tumor cell (e.g., an OvCa cell, a cervical tumor cell, a brain tumor cell, a breast tumor cell, a prostate tumor cell, or a hepatic tumor cell), and for determining predisposition to or treatability of cancer (e.g., OvCa, cervical cancer, brain cancer, breast cancer, prostate cancer, or hepatic cancer) in an individual. In particular, the invention provides materials and methods related to Bex4, a gene that is down regulated in cancer cells (e.g., OvCa cells). Bex4 may be useful for treating cancer patients, as Bex4 expression induces apoptosis and reduces colony formation efficiency. Furthermore, the methylation status of the Bex4 gene may be a useful indicator of a cancer patient's prognosis, as cells expressing Bex4 (e.g., normal ovarian epithelial cells) display lower levels of DNA methylation than cells that do not express Bex4 (e.g., tumor cells).

Isolated Bex4 Nucleic Acid Molecules

The invention provides isolated Bex4 nucleic acid molecules. Such nucleic acids can contain all or part of the coding sequence and/or non-coding sequence from the Bex4 gene. As used herein, the term “nucleic acid” refers to both RNA and DNA, including cDNA, genomic DNA, and synthetic (e.g., chemically synthesized) DNA. The nucleic acid can be double-stranded or single-stranded (i.e., a sense or an antisense single strand). As used herein, “isolated nucleic acid” refers to a nucleic acid that is separated from other nucleic acid molecules that are present in a mammalian genome, including nucleic acids that normally flank one or both sides of the nucleic acid in a mammalian genome (e.g., nucleic acids that flank a Bex4 gene). The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one or both of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules are at least 10 nucleotides in length (e.g., 10, 20, 50, 100, 200, 300, 400, 500, 1000, or more nucleotides in length). As described in the Examples (below), the full-length human Bex4 transcript contains 3 exons, with a coding region that is 300 nucleotides in length. A Bex4 nucleic acid molecule is not required to contain all of the coding region or all of the exons; in fact, a Bex4 nucleic acid molecule can contain as little as a single exon or a portion of a single exon (e.g., 10 nucleotides from a single exon). Nucleic acid molecules that are less than full-length can be useful, for example, for diagnostic purposes.

Isolated nucleic acid molecules of the invention can be produced by standard techniques, including, without limitation, common molecular cloning and chemical nucleic acid synthesis techniques. For example, polymerase chain reaction (PCR) techniques can be used to obtain an isolated Bex4 nucleic acid molecule. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication or nucleic acid sequence-based amplification also can be used to obtain isolated nucleic acids. See, for example, Lewis (1992) Genetic Engineering News 12(9):1; Guatelli et al. (1990) Proc. Natl. Acad. Sci. USA 87:1874-1878; and Weiss (1991) Science 254:1292-1293.

Isolated Bex4 nucleic acid molecules also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Vectors and Host Cells

The invention also provides vectors containing nucleic acids such as those described above. As used herein, a “vector” is a replicon, such as a plasmid, phage, or cosmid, into which another DNA segment may be inserted so as to bring about the replication of the inserted segment. The vectors of the invention can be expression vectors. An “expression vector” is a vector that includes one or more expression control sequences, and an “expression control sequence” is a DNA sequence that controls and regulates the transcription and/or translation of another DNA sequence.

In the expression vectors of the invention, the nucleic acid is operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen Madison, Wis.), Clontech (Palo Alto, Calif.), Stratagene (La Jolla, Calif.), and Invitrogen/Life Technologies (Carlsbad, Calif.).

An expression vector can encode a tag sequence designed to facilitate subsequent manipulation of the expressed nucleic acid sequence (e.g., purification or localization). Tag sequences, such as glutathione S-transferase (GST), polyhistidine, c-myc, hemagglutinin, or FLAG® tag (Kodak, New Haven, Conn.) sequences typically are expressed as a fusion with the encoded polypeptide. Such tags can be inserted anywhere within the polypeptide including at either the carboxyl or amino terminus.

The invention also provides host cells containing vectors of the invention. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Suitable methods for transforming and transfecting host cells also are found in Sambrook et al., Molecular Cloning: A Laboratory Manual (2^{nd} edition), Cold Spring Harbor Laboratory, New York (1989), and reagents for transformation and/or transfection are commercially available (e.g., LIPOFECTIN® (Invitrogen/Life Technologies); FUGENE™ (Roche, Indianapolis, Ind.); and SUPERFECT® (Qiagen, Valencia, Calif.)).

Purified Bex4 Polypeptides

The invention provides purified Bex4 polypeptides that are encoded by the Bex4 nucleic acid molecules of the invention. A “polypeptide” refers to a chain of at least 10 amino acid residues (e.g., 10, 20, 50, 75, 100, or more than 100 residues), regardless of post-translational modification (e.g., phosphorylation or glycosylation). Typically, a Bex4 polypeptide of the invention is capable of eliciting a Bex4-specific antibody response (i.e., is able to act as an immunogen that induces the production of antibodies capable of specific binding to Bex4).

A Bex4 polypeptide can have an amino acid sequence that is identical to at least a portion of SEQ ID NO:1. Alternatively, a Bex4 polypeptide can include an amino acid sequence variant. As used herein, an amino acid sequence variant refers to a deletion, insertion, or substitution. For example, a Bex4 polypeptide can contain amino acid substitutions at up to twenty amino acid positions (e.g., one, two, three, four, five, six, seven, eight, nine, ten, 11, 12, 13, 14, 15, 16, 17, 18, 19, or 20 positions) relative to the full-length amino acid sequence set forth in SEQ ID NO:1. In another embodiment, a Bex4 polypeptide can be a fusion polypeptide that contains a Bex4 amino acid sequence linked to an amino acid tag (e.g., FLAG®, His, or c-myc), or to another polypeptide such as green fluorescent protein (GFP). In yet another embodiment, a Bex4 polypeptide can contain an amino acid sequence that is a fragment of that set forth in SEQ ID NO:1. A fragment can contain, for example, from about 25 to about 95 amino acid residues (e.g., about 25, 30, 40, 50, 60, 70, 80, 85, 90, or about 95 amino acid residues).

The term “purified” as used herein with reference to a polypeptide refers to a polypeptide that either has no naturally occurring counterpart (e.g., a peptidomimetic), has been chemically synthesized and is thus uncontaminated by other polypeptides, or has been separated or purified from other cellular components by which it is naturally accompanied (e.g., other cellular proteins, polynucleotides, or cellular components). Typically, a polypeptide is considered “purified” when it is at least 70% (e.g., 70%, 80%, 90%, 95%, or 99%), by dry weight, free from the proteins and naturally occurring organic molecules with which it naturally associates.

Bex4 polypeptides can be produced by a number of methods, many of which are well known in the art. By way of example and not limitation, Bex4 polypeptides can be obtained by extraction from a natural source (e.g., from isolated cells, tissues or bodily fluids), by expression of a recombinant nucleic acid encoding the polypeptide, or by chemical synthesis.

Bex4 polypeptides of the invention can be produced by, for example, standard recombinant technology, using expression vectors encoding Bex4 polypeptides. The resulting Bex4 polypeptides then can be purified. Expression systems that can be used for small or large scale production of Bex4 polypeptides include, without limitation, microorganisms such as bacteria (e.g., E. coli and B. subtilis) transformed with recombinant bacteriophage DNA, plasmid DNA, or cosmid DNA expression vectors containing the nucleic acid molecules of the invention; yeast (e.g., S. cerevisiae) transformed with recombinant yeast expression vectors containing the nucleic acid molecules of the invention; insect cell systems infected with recombinant virus expression vectors (e.g., baculovirus) containing the nucleic acid molecules of the invention; plant cell systems infected with recombinant virus expression vectors (e.g., tobacco mosaic virus) or transformed with recombinant plasmid expression vectors (e.g., Ti plasmid) containing the nucleic acid molecules of the invention; or mammalian cell systems (e.g., primary cells or immortalized cell lines such as COS cells, Chinese hamster ovary cells, HeLa cells, human embryonic kidney 293 cells, and 3T3 L1 cells) harboring recombinant expression constructs containing promoters derived from the genome of mammalian cells (e.g., the metallothionein promoter) or from mammalian viruses (e.g., the adenovirus late promoter and the cytomegalovirus promoter), along with the nucleic acids of the invention.

Suitable methods for purifying the polypeptides of the invention can include, for example, affinity chromatography, immunoprecipitation, size exclusion chromatography, and ion exchange chromatography. See, for example, Flohe et al. (1970) Biochim. Biophys. Acta. 220:469-476, or Tilgmann et al. (1990) FEBS 264:95-99. The extent of purification can be measured by any appropriate method, including but not limited to: column chromatography, polyacrylamide gel electrophoresis, or high-performance liquid chromatography. Bex4 polypeptides also can be “engineered” to contain a tag sequence described herein that allows the polypeptide to be purified (e.g., captured onto an affinity matrix). Immunoaffinity chromatography also can be used to purify Bex4 polypeptides.

Methods of Using Bex4 Nucleic Acids and Polypeptides

The invention provides methods for using Bex4 nucleic acid molecules and Bex4 polypeptides to treat individuals with cancer (e.g., OvCa, breast cancer, cervical cancer, brain cancer, liver cancer, or prostate cancer). For example, a vector containing a Bex4 nucleic acid sequence that encodes a Bex4 polypeptide can be administered to a tumor cell, such that expression of the encoded Bex4 polypeptide can induce apoptosis and kill the tumor cell. Suitable methods for introducing nucleic acids into cells include those known in the art, such as the transfection and transformation techniques disclosed above. Any other suitable method of transferring a nucleic acid molecule into a cell (e.g., viral transformation) also can be used.

In another embodiment, a Bex4 polypeptide (e.g., a Bex4 polypeptide having the amino acid sequence of SEQ ID NO:1 or a fragment thereof) can be administered directly to a tumor cell in a mammal (e.g., in a human cancer patient) in order to kill the cell. Alternatively, a Bex4 polypeptide or nucleic acid can be used in the manufacture of a medicament for killing a tumor cell. In some embodiments, implantable medical devices can be used to deliver Bex4 polypeptides to a mammal, and in particular to a human patient. For example, Bex4 polypeptides can be incorporated into a coated device such that the polypeptides are eluted over time. Alternatively, a medical device can be seeded with cells such as smooth muscle cells, fibroblasts, hepatocytes, ovarian cells, epithelial cells, endothelial cells, or stem cells in vitro, and then implanted into a patient. Typically, cells are harvested from the patient in whom the medical device will be implanted. In some embodiments, however, cells can be harvested from a donor of the same or of a different species that is not the recipient of the medical device. For example, it may be useful to harvest cells from a pig for transplantation into a human. Cells that are seeded onto the medical device can be modified such that the cells produce Bex4 polypeptides. Such polypeptides can be secreted into the vasculature, for example. Implantable medical devices thus can deliver a Bex4 polypeptide to a mammal for treating cancer (e.g., OvCa, breast cancer, cervical cancer, brain cancer, liver cancer, or prostate cancer).

To modify isolated cells such that a Bex4 polypeptide is produced, the appropriate exogenous nucleic acid must be delivered to the cells. Primary cultures or secondary cell cultures can be modified and then seeded onto an implantable device. In some embodiments, transient transformants in which the exogenous nucleic acid is episomal (i.e., not integrated into the chromosomal DNA), can be seeded onto a medical device. Typically, stable transformants in which the exogenous nucleic acid has integrated into the host cell's chromosomal DNA are selected. The term “exogenous” as used herein with reference to a nucleic acid and a particular cell refers to any nucleic acid that does not originate from that particular cell as found in nature. In addition, the term “exogenous” includes a naturally occurring nucleic acid. For example, a nucleic acid encoding a polypeptide that is isolated from a human cell is an exogenous nucleic acid with respect to a second human cell once that nucleic acid is introduced into the second human cell.

An exogenous nucleic acid can be transferred to cells within a primary or secondary culture using recombinant viruses that can infect cells, or liposomes or other non-viral methods such as electroporation, microinjection, or calcium phosphate precipitation. An exogenous nucleic acid that is delivered typically is part of a vector in which a regulatory element such as a promoter is operably linked to the nucleic acid of interest. The promoter can be constitutive or inducible. Non-limiting examples of constitutive promoters include the cytomegalovirus (CMV) promoter and the Rous sarcoma virus (RSV) promoter. As used herein, “inducible” refers to both up-regulation and down regulation. An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent such as a protein, metabolite, growth regulator, phenolic compound, or a physiological stress imposed directly by, for example heat, or indirectly through the action of a pathogen or disease agent such as a virus. The inducer also can be an illumination agent such as light and light's various aspects, which include wavelength, intensity, fluorescence, direction, and duration.

An example of an inducible promoter is the tetracycline (tet)-on promoter system, which can be used to regulate transcription of the nucleic acid. In this system, a mutated Tet repressor (TetR) is fused to the activation domain of herpes simplex VP16 (transactivator protein) to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A.

Additional regulatory elements that may be useful in vectors, include, but are not limited to, polyadenylation sequences, translation control sequences (e.g., an internal ribosome entry segment, IRES), enhancers, or introns. Such elements may not be necessary, although they may increase expression by affecting transcription, stability of the mRNA, translational efficiency, or the like. Such elements can be included in a nucleic acid construct as desired to obtain optimal expression of the nucleic acids in the cell(s). Sufficient expression, however, may sometimes be obtained without such additional elements.

Other elements that can be included in vectors include nucleic acids encoding selectable markers. Non-limiting examples of selectable markers include puromycin, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture.

Viral vectors also can be used. Suitable viral vectors include, for example, adenovirus, adeno-associated virus (AAV), retroviruses, lentiviruses, vaccinia virus, measles viruses, herpes viruses, and bovine papilloma virus vectors. See, Kay et al. (1997) Proc. Natl. Acad. Sci. USA 94:12744-12746 for a review of viral and non-viral vectors. Viral vectors are modified so the native tropism and pathogenicity of the virus has been altered or removed. The genome of a virus also can be modified to increase its infectivity and to accommodate packaging of the nucleic acid encoding the polypeptide of interest.

Adenoviral vectors can be easily manipulated in the laboratory, can efficiently transduce dividing and nondividing cells, and rarely integrate into the host genome. Smith et al. (1993) Nat. Genet. 5:397-402; and Spector and Samaniego (1995) Meth. Mol. Genet. 7:31-44. The adenovirus can be modified such that the E1 region is removed from the double stranded DNA genome to provide space for the nucleic acid encoding the polypeptide and to remove the transactivating E1 a protein such that the virus cannot replicate. Adenoviruses have been used to transduce a variety of cell types, including, inter alia, keratinocytes, hepatocytes, and epithelial cells.

Adeno-associated viral (AAV) vectors demonstrate a broad range of tropism and infectivity, although they exhibit no human pathogenicity and do not elicit an inflammatory response. AAV vectors exhibit site-specific integration and can infect non-dividing cells. AAV vectors have been used to deliver nucleic acid to brain, skeletal muscle, and liver over a long period of time (e.g., >9 months in mice) in animals. See, for example, U.S. Pat. No. 5,139,941 for a description of AAV vectors.

Retroviruses are the most-characterized viral delivery system and have been used in clinical trials. Retroviral vectors mediate high nucleic acid transfer efficiency and expression. Retroviruses enter a cell by direct fusion to the plasma membrane and integrate into the host chromosome during cell division.

Lentiviruses also can be used to deliver nucleic acids to cells, and in particular, to non-dividing cells. Replication deficient HIV type I based vectors have been used to transduce a variety of cell types, including stem cells. See, Uchidda et al. (1998) Proc. Natl. Acad. Sci. USA 95:11939-11944.

Non-viral vectors can be delivered to cells via liposomes, which are artificial membrane vesicles. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Transduction efficiency of liposomes can be increased by using dioleoylphosphatidyl-ethanolamine during transduction. See, Felgner et al. (1994) J. Biol. Chem. 269:2550-2561. High efficiency liposomes are commercially available. See, for example, SUPERFECT® from Qiagen.

In another embodiment, the invention provides methods for determining whether an individual is predisposed to develop cancer (e.g., OvCa, breast cancer, cervical cancer, brain cancer, liver cancer, or prostate cancer). A method can involve, for example, measuring the amount of Bex4 mRNA or protein in a biological sample (e.g., ovarian cells) obtained from an individual, and comparing the amount of mRNA or protein to, for example, an amount determined from an individual known to have such a cancer, an individual who does not have such a cancer, or an average amount determined from measuring Bex4 mRNA or protein in a population of individuals who have or do not have such a cancer. If the amount of Bex4 mRNA or protein in ovarian cells from the individual is lower than the amount in cells obtained from a normal individual (i.e., an individual who does not have OvCa), then the individual in question may be predisposed to develop OvCa. Alternatively, if the amount of Bex4 mRNA or protein in ovarian cells from the individual is higher than the amount in cells obtained from an OvCa patient, then the individual in question may not be predisposed to develop OvCa.

The invention also provides methods for detecting cancer recurrence (e.g., in an individual with OvCa). Methods can include measuring the level of methylation of the Bex4 gene in cells obtained from a cancer patient, and comparing the level to a baseline level of methylation (e.g., the level of Bex4 methylation in cells obtained from a normal individual who does not have cancer). For example, cells can be obtained from a peritoneal washing of an individual who has been treated for cancer (e.g., an individual treated for OvCa and undergoing second look laparoscopy or laparotomy (SLL)), and the degree of Bex4 hypermethylation can be determined as discussed in Example 6. As used herein, “hypermethylation” means that the Bex4 gene is more highly methylated in one individual (e.g., an OvCa patient) than in another individual (e.g., a normal individual). The presence or absence of hypermethylation can be used as an indicator of the presence or absence, respectively, of cancer cells in an individual (e.g., an OvCa patient). In turn, the presence or absence of cancer cells in the individual can indicate whether or not the cancer has recurred.

The invention will be further described in the following examples, which do not limit the scope of the invention described in the claims.

### EXAMPLES

### Example 1

### Materials and Methods for Identification of OvCa Genes

Tissue Processing: All specimens were snap-frozen in the surgical pathology unit at the Mayo Clinic. The tumor content of the specimens was assessed by hematoxylin and eosin-stained sections. Only specimens with >75% tumor content were used for all of the experiments. Twenty normal ovarian epithelial cell brushings from patients without cancer were pooled, and the epithelial nature of these brushings was verified by cytokeratin staining. Only brushings that contained >90% epithelial cell content were used. The majority of patients providing normal ovaries were between 45 and 65 years old and were undergoing incidental oophorectomy at the time of pelvic surgery for other indications. All of the ovaries were examined pathologically and found to be benign. Tumors were staged according to the criteria proposed by International Federation of Gynecology and Obstetrics.

Cell Culture: Five of seven ovarian-carcinoma cell lines (OV167, OV177, OV202, OV207, and OV266) were low-passage primary lines established at the Mayo Clinic (Conover et al. (1998) Exp. Cell Res. 238:439-449). SKOV-3 was purchased from the American Type Culture Collection (ATCC; Manassas, Va.). OVCAR 5 is a NIH human OvCa cell line (Hamilton et al. (1984) Semin. Oncol., 11:285-298). All cells were grown according to the supplier's recommendations.

Suppression Subtraction Libraries: Four down-regulated libraries were generated from individual tumors. OV338 (stage I endometrioid), OV402 (stage II serous), and two stage III serous tumors (OV4 and OV13) were subtracted against normal ovarian epithelial cell brushings.

Tester and Driver Preparations: Total cellular RNA from primary ovarian tumors (driver) and from 20 pooled normal ovarian epithelial cell brushings (tester) was prepared using TRIZOL® reagent (Invitrogen/Life Technologies) followed by purification using an RNEASY® kit (Qiagen). The integrity of the RNA was assessed by agarose gel electrophoresis. One μg of total RNA was then used for first- and second-strand cDNA synthesis in a 10 μl reaction volume using Smart II oligonucleotides and cDNA synthesis (CDS) primer (Clontech, Palo Alto, Calif.) following the manufacturer's instructions. The concentration of reverse-transcribed cDNA was adjusted to 25 ng/μl. The resulting cDNAs were amplified, and the cycle number was optimized for each sample after amplification with PCR primer (5′-AAGCAGTGGTAACAACGCAGAGT-3′; SEQ ID NO:10). For cycle optimization, aliquots of the PCR reactions were removed after 15, 18, 21, and 24 cycles of amplifications. The resulting products were resolved on a 1.5% agarose gel, and optimum cycle number was chosen after southern hybridization with GAPDH and transferrin receptor genes as probes. For most samples, the optimum cycle numbers were between 17 and 19 cycles of amplification. The reaction was scaled up to generate 3 μg of double stranded cDNAs. The resulting cDNA was precipitated, washed with 70% ethanol, dissolved in 40 μl of deionized water, and digested with RsaI in a 50-μl reaction mixture containing 100 units of enzyme (Boeringher Mannheim, Indianopolis, Ind.) for 3 hours. The blunt-ended cDNAs were then purified using PCR purification columns (Promega, Madison, Wis.). The driver cDNAs from primary tumors were adjusted to 300 ng/μl in a final 7-μl volume. Fifty ng of digested double-stranded tester (normal ovarian epithelial cell brushings) cDNA was ligated in two separate reactions with 2 μl of adapter 1 (10 μM) and adapter 2 (10 μM; provided in the kit), respectively, and 1.0 unit of T4 DNA ligase (Invitrogen/Life Technologies) in a 10-μl total volume with buffer supplied by the manufacturer. After ligation, 1 μl of 0.2M EDTA was added and the samples were heated at 75° C. for 5 minutes to inactivate the ligase and stored at −20° C.

Subtractive Hybridization: SSH was performed between tester and driver mRNA populations using the PCR-select cDNA subtraction kit (Clontech) according to the manufacturer's recommendations. Two μl of driver double stranded cDNA (150-200 ng/μl) was added to each of two tubes containing one μl of adapter-1 and adapter-2 ligated tester cDNA (10 ng) with 1×hybridization buffer in a total volume of 4 μl. The solution was overlaid with 10 μl of mineral oil and the cDNAs were denatured (1.5 minutes, 98° C.) and allowed to anneal for 8-9 hours at 68° C. After the first hybridization, the two samples were combined and an additional heat-denatured driver (300 ng) in 1×hybridization buffer was added. The sample was allowed to hybridize for another 16 hours at 68° C. The final hybridization reaction was diluted with 200 μl of dilution buffer provided by the manufacturer, heated at 68° C. for 7 minutes, and stored at −20° C.

PCR Amplification: For each subtraction, two PCR amplifications were performed. The primary PCR reaction in 25 μl contained 1 μl of subtracted cDNA, 1 μl of PCR primer1 (10 μM, 5′-CTAATACGACTCACTATGGGC-3′; SEQ ID NO:11), and 0.5 μl each of 50×Advantage cDNA polymerase mix (Clontech) and 10 mM dNTP mix. The cycling parameters were 75° C. for 7 minutes, followed by 27 cycles at 94° C. for 30 seconds, 68° C. for 30 seconds, and 72° C. for 2 minutes. The amplified products were diluted 10-fold with deionized water and 2 μl were used in the secondary PCR reactions with NP1 and NP2 primers (provided in the kit). The cycling conditions were the same as in the primary PCR amplification, except the reactions were in a 50-μl volume for 11 cycles only, with a final extension cycle for 7 minutes at 68° C. The subtraction efficiency was determined by both PCR and Southern based methods as instructed by the manufacturer.

Cloning and Analysis of the Subtracted cDNAs: Products from the secondary PCRs were inserted into PCR2.1-TOPO TA cloning kit (Invitrogen/Life Technologies) following the manufacturer's instructions. Prior to ligation, the subtracted cDNA mix was incubated for 1 hour at 72° C. with dATP and AmpliTaq DNA polymerase (Perkin-Elmer Cetus, Foster City, Calif.) to ensure that most of the cDNA fragments contained “A” overhangs. Approximately 100 ng of PCR amplified cDNA were ligated into 50 ng of vector without further purification. Two μl of ligated products (10 ng of vector and 50 ng of cDNAs ligated in 10 μl volume) were transformed into 40 μl of DH10B cells by electroporation (Bio-Rad Laboratories, Hercules, Calif.). Routinely, 50- and 200-μl aliquots of the transformed cells (grown in 1 ml of medium) were plated onto 150-mm Luria-Bertani/agar plates containing 100 μg/ml of ampicillin, with 100 μM isopropyl-1-thio-β-D-galactopyranoside (IPTG) and 50 μg/ml 5-bromo-4-chloro-3-indolyl-β-D-galactopyranoside (X-Gal) to discriminate white from blue colonies. The transformation efficiency was 2−4×10⁶ colonies/μg of DNA.

Hybridization and Screening for Differentially Expressed Transcripts: The differential hybridization was performed initially on 96 randomly picked clones to determine subtraction efficiency. The inserts in the plasmid were amplified using NP-1 and NP-2 primers provided in the kit. The PCR conditions were 94° C. for 4 minutes followed by 30 cycles of 94° C. for 30 seconds, 60° C. for 30 seconds, and 72° C. for 2 minutes, followed by a final extension at 72° C. for 5 minutes. The products of the PCR reactions were resolved on a 2% agarose gel run in duplicate. After Southern blotting of the amplified inserts onto Hybond N membranes (Amersham, Piscataway, N.J.), the membranes were stained with methylene blue in 0.2×SSC and visualized to ensure complete transfer of all of the products. The blots were then hybridized with RsaI-digested cDNA probes (reverse Northern). Fifty ng of RsaI-digested tester and driver cDNAs were labeled using a random primer labeling kit (Stratagene, LA Jolla, Calif.) with 50 μCi of [³³P]dCTP following the manufacturer's instructions. Equal counts (1-2×10⁷ cpm/μl) of the cDNA probes from normal and tumor tissues were heat-denatured and used to probe duplicate blots. Hybridization was performed at stringent conditions in 0.5 M Na₂PO₄ (pH 7.2), 7% SDS at 65° C. The next day, the filters were washed twice in 2×SSC, 0.5% SDS at 68° C., then once in 0.1% SSC, 0.1% SDS at 68° C., and exposed to phosphorimager screens overnight. The signal intensity of each spot in the membranes was compared between tester and driver hybridized duplicates. cDNA fragments displaying differential expression levels of >1.8-fold or higher were selected to estimate the efficiency of the differential hybridization.

Approximately 600-700 unique clones from each of the four libraries were successfully sequenced with M13 forward primer using an ABI Prism dye terminator cycle sequencing in the sequencing core at Millennium Predictive Medicine, Cambridge, Mass. Sequences were compared with the National Center for Biotechnology Information sequence database using the BLAST program.

Semiquantitative RT-PCR: Fifty to 100 ng of reverse-transcribed cDNAs were used in a multiplex reaction with a pair of gene-specific primers and GAPDH forward (5′-ACCACAGTCCATGCCATCAC-3′; SEQ ID NO:12) and reverse primers (5′-TCCACCACCCTGTTGCTTGTA-3′; SEQ ID NO:13), which yielded a 450-bp product. The PCR reaction mixes contained 50 mM Tris-HCl (pH 8.3), 1.5 mM MgCl₂, 400 μM gene-specific primers, 50 μM each of the GAPDH primers, and 0.5 units of Taq polymerase (Promega), in a 12.5 μl reaction volume. The conditions for amplification were as follows: 94° C. for three minutes, then 29 cycles of 94° C. for 30 seconds, 50-62° C. for 30 seconds depending on the genespecific primers being tested, and 72° C. for 30 seconds in a Perkin-Elmer-Cetus 9600 Gene-Amp PCR system. The products of the reactions were resolved on a 1.6% agarose gel. Band intensities were quantified using the Gel Doc 1000 photo-documentation system (Bio-Rad) and its associated software.

The following gene-specific primers were used: for cathepsin K (CTSK), (forward) 5′-GGAGATACTGGACAACCCACTG-3′ (SEQ ID NO:14) and (reverse) 5′-CCAACTCCCTTCCAAAGTGC-3′ (SEQ ID NO:15); for Plasminogen activator inhibitor 1 (PAI1), (forward) 5′-AATCGCAAGGCACCTCTGAG-3′ (SEQ ID NO:16) and (reverse) 5′-GATCTGGTTTACCATCTTTT-3′ (SEQ ID NO:17); for cyclin D2, (forward) 5′-AGCTGCTGTGCCACGAGGT (SEQ ID NO:18) and (reverse) 5′-ACTGGCATCCTCACAGGTC (SEQ ID NO:19); for fibroblast growth factor 7 (FGF7), (forward) 5′-TAATGCACAAATGGATAC (SEQ ID NO:20) and (reverse) 5′-ATTGCCATAGGAAGAAAG -3′ (SEQ ID NO:21); for early growth response 1 (EGR1), (forward) 5′-GACACCAGCTCTCCAGCCTGC-3′ (SEQ ID NO:22) and (reverse) 5′-GGAAGGGCTTCTGGTCTGGGG-3′ (SEQ ID NO:23); for secreted protein acidic and rich in cysteine (SPARC), (forward) 5′-CCACTGAGGGTTCCCAGCAC-3′ (SEQ ID NO:24) and (reverse) 5′-GGAAACACGAAGGGGAGGGT-3′ (SEQ ID NO:25); for decorin, (forward) 5′-CCTGGTTGTGAAAATACATGA-3′ (SEQ ID NO:26) and (reverse) 5′-TGACATTAACAAGATTTTGCC-3′ (SEQ ID NO:27); for thrombospondin 2 (THBS2), (forward) 5′-TGGTCACCAGGACAAAGACAC-3′ (SEQ ID NO:28) and (reverse) 5′-ATCCTGCCAGCAAGCTGACA-3′ (SEQ ID NO:29); for integral membrane protein 2a (ITM2A), (forward) 5′-CGCAGCCCGAAGATTCACTATG-3′ (SEQ ID NO:30) and (reverse) 5′-CTTATTACCAAGGACACTCTATCT-3′ (SEQ ID NO:31); and for paternally expressed gene 3 (PEG3), (forward) 5′-CGGAGAACTGTGAGAAGCTCGTC-3′ (SEQ ID NO:32) and (reverse) 5′-GGTGGGGCTAGGCTAGAAGG-3′ (SEQ ID NO:33).

Northern Blot Analysis: Fifteen μg of total RNA was fractionated on 1.2% formaldehyde agarose gels and blotted in 1×SPC buffer [10 mM sodium phosphate (pH 6.8), 1 mM CDTA (Sigma Chemical Co., St. Louis, Mo.)] onto Hybond N membranes (Amersham, Piscataway, N.J.). The control small ribosomal protein S9, (RPS9) and gene-specific probes were labeled using a random primer labeling system (Invitrogen/Life Technologies) and purified using spin columns (TE-100) from Clontech. Filters were hybridized at 68° C. with radioactive probes in a microhybridization incubator (Model 2000, Robbins Scientific, Sunnyvale, Calif.) for 1-3 hours in Express hybridization solution (Clontech) and washed according to the supplier's guidelines. The primers, (forward) 5′-GCAACATGCCAGTGGCCCGG-3′ (SEQ ID NO:34) and (reverse) 5′-ATCCTCCTCCTCGTCGTCTC-3′ (SEQ ID NO:35) for RPS9 yielded a 586-bp cDNA fragment, and the conditions for amplification were similar to the semiquantitative RT-PCR conditions described above.

LOH Analysis: Fifteen early- and 18 late-stage tumors of differing histologies were analyzed. The 15 early-stage tumors included 3 clear-cell, and 6 each of endometrioid and serous tumors. The 18 late-stage tumors included 3 clear-cell, 4 endometrioid, and 12 serous tumors. The markers used in this study were obtained from Research Genetics (Huntsville, Ala.). Two new microsatellite markers, one near Methylation Controlled J (MCJ) protein on 13q14.1 (Shridhar et al. (2001) Cancer Res. 61:4258-4265) and another marker within BAC CIT-B-470f8 on 19q14.3 (AC006115) were amplified with the following primers: MCJ-NF: 5′-GATTGACCACAGTCTTATCT-3′ (SEQ ID NO:36) and MCJ-18: 5′-TAAGAGGTCTACTCATTGCTCAC-3′ (SEQ ID NO:37), and 19-F: 5′-GCACCTGGCCCAACTGTAAC-3′ (SEQ ID NO:38) and 19-R: 5′-CCAGCTGCTGGCTCACCTT-3′ (SEQ ID NO:39), respectively. The individual oligonucleotides were synthesized in the Mayo Molecular Technology Core at the Mayo Clinic, Rochester, Minn. The PCR mix contained 50 ng of genomic DNA, 50 mM KCl, 10 mMTris-HCl (pH 8.3), 1.5 mM MgCl₂, 200 μM concentration of each primer, 0.05 μl of [³²P]dCTP (10 μCi/μl), and 0.5 units of Taq polymerase (Promega) in a 10 μl reaction volume. The conditions for amplification were: 94° C. for two minutes, then 30 cycles of 94° C. for 30 seconds, 52-57° C. for 30 seconds, and 72° C. for 30 seconds in a Perkin-Elmer-Cetus 9600 Gene-Amp PCR system in a 96-well plate. PCR products were denatured and run on 6% polyacrylamide sequencing gels containing 8 M urea. The gels were dried and autoradiographed for 16-24 hours and scored for LOH. Multiple exposures were used before scoring for LOH. Allelic imbalance indicative of LOH was scored when there was more than 50% loss of intensity of one allele in the tumor sample with respect to the matched allele from normal tissue. The evaluation of the intensity of the signal between the different alleles was determined by visual examination by two independent viewers.

### Example 2

### Identification of Genes that are Underexpressed in OvCa

To identify novel tumor suppressor genes in OvCa, down-regulated cDNA libraries were generated from two early-stage (stages I and II; OV338 and OV402) and two late-stage (stage III; OV4 and OV13) tumors subtracted against normal ovarian epithelial cell brushings, as described in Example 1. The libraries were monitored at each stage of library construction to ensure that the clones generated from each of the four libraries truly reflected differentially expressed sequences. The subtraction efficiency was determined by both Southern- and PCR-based protocols. A 60- to 70-fold enrichment of the differentially expressed genes was estimated in all four libraries. This was confirmed with the Southern-based analysis, a complete subtraction of GAPDH was observed in the subtracted cDNAs.

The differential expression of genes was evaluated in each of the libraries by hybridizing tester and driver cDNAs to randomly amplify 96 cloned inserts by colony PCR. PCR products were resolved in duplicate. Care was taken to ensure equal loading of the PCR products onto 2% agarose gels to allow direct comparison of hybridization signal intensities. After transfer of the PCR products onto nylon membranes, reverse Northern analyses were performed to identify differentially expressed transcripts. The cDNA probes used for hybridization were restricted with RsaI to minimize background hybridization. Faint signals representing rare transcripts were easily distinguished with this approach. After densitometric analysis of each of the corresponding bands hybridized with tester and driver cDNAs, the percentage of these clones that showed the expected differential hybridization was 70-80%.

About 2000 randomly picked clones were sequenced from each of the four libraries. After consolidating for clones that appeared more than once in the libraries, there were about 600 distinct clones sequenced from each of the four libraries.

Analysis of SSH Library Gene: To discern the differences in gene expression in early- versus late-stage tumors, the sequenced genes were compared to verify how many were in common among the four libraries. Of the 600 or so distinct clones in each library, 45 genes were common to all four libraries. These potentially represented genes consistently down regulated in both early- and late-stage ovarian tumors. A similar comparison of genes that were isolated from any three of the four libraries revealed 80 common genes. There were 130 common genes in the two early-stage tumors. Sixty of these also were present in one of the two late-stage tumors. A similar analysis comparing sequences in the two late-stage libraries revealed that there were 210 genes in common between them. Only 55 of these also were identified in either one of the two libraries generated from early-stage tumors.

Because the sequenced clones were randomly selected, the differential expression of 20 genes was validated. These genes ranged from clones that were highly represented to those that occurred infrequently in the libraries, to ensure that the generated sequences truly were representative of genes that are differentially expressed between normal and tumor cells. Initially, seven ovarian tumor cell lines were used for validation by semiquantitative RT-PCR, with GAPDH as a control. The expression profile of these genes in tumor cell lines was compared with short term cultures of normal ovarian epithelial cells. Several of these genes showed a complete loss of expression in a number of cell lines. For example, HSD3B1 (Morisette et al. (1995) Cytogenet. Cell Genet. 69:59-62), which was represented by only two clones in each of the four libraries, showed complete loss of expression in all of the seven tested cell lines. However, PAI1 (Bajou et al. (1998) Nat. Med. 4:923-928), which appeared several times (100-140) in each of the two late-stage libraries, showed complete loss of expression in only two of seven cell lines. The differential expression of these genes also was examined in 20 early (I/II)-stage and 16 late (III/IV)-stage primary tumors of mixed histological subtypes, using semiquantitative RT-PCR to compare them with normal epithelial cell brushings. The 20 early-stage tumors included 5 clear cell, 6 endometrioid, and 9 serous tumors. The late-stage tumors included 1 clear cell, 4 endometrioid, and 11 serous tumors.

In addition, the expression of HSD3B1 and PRSS11 (a serine protease with an insulin-like growth-factor-binding domain; Zumbrunn et al. (1997) Genomics 45:461-462) was tested in cell lines and primary tumors by Northern analysis. HSD3B1 showed complete loss of expression in all of the cell lines and primary tumors. PRSS11 showed complete loss of expression in four of seven cell lines and in three of eight primary tumors. Lower levels of PRSS11 expression also was detected in four of eight primary tumors. Control probe RPS9 was hybridized to the cell line and primary tumor blots, indicating equal loading of RNA.

Chromosomal Sorting of SSH Genes: Genes from each of the four libraries were sorted based on their chromosomal positions. Several of the common genes identified in three or all four libraries mapped to known regions of deletions in OvCa (Allan et al. (1994) Hum. Mutat. 3:283-291; Cliby et al. (1993) Cancer Res. 53:2393-2398; Shridhar et al. (1999) Oncogene 18:3913-3918; and Bicher et al. (1997) Gynecol. Oncol. 66:36-40). For example, ARHI (NOEY2), a well characterized imprinted tumor suppressor gene, with a reported LOH in 40-50% of OvCa cases, maps to 1p31 (Yu et al. (1999) Proc. Natl. Acad. Sci. USA 96:214-219). In addition, caveolin 1 (Engelman et al. (1998) FEBS Lett. 436:403-410), which maps to 7q31.1-31.2 (a known region of deletion in OvCa; Huang et al. (1999) Genes Chromosomes Cancer 24:48-55), was identified in all four libraries. Other genes also mapped to specific chromosomal regions of deletions in OvCa. Of interest are chromosomal bands 5q31-32, 10q11, and 10q25.3-26.2, because several of the down-regulated genes isolated from these bands were common to three, or were in all four, of the libraries. The 5q31-32 genes were catenin (Bugert et al. (1998) Int. J Cancer 76:337-340), FGF1 (Crickard et al. (1994) Gynecol. Oncol. 55:277-284), HDAC3 (Wen et al. (2000) Proc. Natl. Acad. Sci. USA 97:7202-7207), selenoprotein P, plasma1 (SEPP1; Holben et al. (1999) J. Am. Diet. Assoc. 99:836-843), testican (SPOCK; Charbonnier et al. (1998) Genomics 48:377-380), transcription elongation factor B (SIII) polypeptide-like (TCEB1L; Conaway et al. (1993) Cell. Mol. Biol. Res. 39:323-329), transforming growth factor, β-induced, Mᵣ 68,000 (TGFβ1; Cardillo et al. (1997) J. Exp. Clin. Cancer Res. 16:49-56), CDC23, (Zhao et al. (1998) Genomics 53:184-190), EGR1 (Du et al. (2000) J. Biol. Chem. 275:39039-39047), and osteonectin (SPARC; Brown et al. (1999) Gynecol. Oncol. 75:25-33). Down-regulated genes from chromosomal bands 10q11.2 and 10q25.3-26.2 that were identified from all four libraries were annexin A8 (ANXA8; Liu et al. (2000) Br. J. Cancer 83:1473-1479) and PRSS11 (Hu et al. (1998) J. Biol. Chem. 273:34406-34412), respectively. Other genes such as nuclear receptor coactivator 4 (ELE1, 10q11.2; Klugbauer et al. (1998) Oncogene 16:671-675), proteoglycan, secretory granule (PRG1, 10q22.1; Mattei et al. (1989) Hum. Genet. 82:87-88), vinculin (VCL, 10q22.1-23;Mulligan et al. (1992) Genomics, 13:1347-1349), lipase A (LIPA, 10q23.3; Anderson et al. (1991) J. Biol. Chem. 266:22479-22484), and protein phosphatase regulatory (inhibitor) subunit 5 (PPP1R5, 10q23-24; Permana et al. (1999) Biochem. Biophys. Res. Commun. 258:184-186) were identified only from the two late-stage libraries, OV4 and OV13.

LOH Analysis of Chromosomal Regions 1p, 6q, 7q, 8p, 9p, 10q, 13q, 17p, and 19q in Stage I/II and Stage III/IV tumors: Because many of the genes identified from the SSH libraries mapped to known regions of deletions in OvCa, a set of early- and late-stage tumors was analyzed for LOH in regions of the genome to which some of the down-regulated genes mapped. Down regulated genes mapping to chromosomal regions of loss identified from the libraries were HSD3B1, EGR1, serum glucocorticoid kinase (SGK; Brennan et al. (2000) Mol. Cell. Endocrinol. 166:129-136), and forkhead (Drosophila) homologue 1 (rhabdomyosarcoma; FRKH; Davis et al. (1995) Hum. Mol. Genet. 4:2355-2362) mapping to 1p12-13, 5q31.1-31.2, 6q23.3, and 13q14.1, respectively. The chromosome 1p11-13 and 6q 23.3 markers showed a higher frequency of loss in late stage-tumors compared with early-stage tumors. Other markers on chromosomes 8, 9, and 10 also showed more losses in high-stage tumors. However, two markers on 5q31 and 13q14.1 and a marker within the BAC CIT-B-470f8 100-kb distal to the PEG3 locus on 19q13.4 had a higher frequency of LOH in early compared with late-stage tumors. Markers D1S440, D1S534, D6S377, and D19S572 showed no LOH in early-stage tumors. To test whether loss, and/or lower levels, of expression of a gene corresponded to a region of loss, a microsatellite repeat within intron 2 of a novel gene on 8q identified in this study was used to determine the frequency of LOH in these tumors. This marker showed 50% loss both in low- and high-stage tumors. There was a direct correlation between lower levels of expression of this gene and loss of an allele by LOH (tumor numbers 684 and 208). In tumors with complete loss of expression and deletion of one of the alleles by LOH (tumor number 182), the remaining allele could be inactivated either by hypermethylation or by transcriptional inactivation as a result of other mechanisms. Tumors without LOH (tumors numbers 13 and 234) showed no loss of expression, as evidenced by semiquantitative RT-PCR.

Thus, LOH analysis revealed known and novel regions of loss to which down regulated genes identified from the SSH libraries map, lending support to the strength of the SSH technique to identify genes with low levels of expression in tumors. Some of these genes could potentially represent candidate tumor suppressor genes involved in ovarian carcinogenesis.

### Example 3

### Identification of an OvCa Gene

Differential display-PCR, cDNA microarray and SHH analyses were used to identify early genetic alterations associated with OvCa, as described in Examples 1 and 2. An EST within the Unigene cluster -Hs.21861 was identified as a down regulated gene in three of four suppression subtraction libraries of primary ovarian tumors subtracted against normal ovarian epithelial cell brushings (OSEs) (Shridhar et al. (2002) Cancer Res. 62:262-270). cDNA microarray analysis of early and late stage ovarian tumors versus normal OSEs also revealed that this EST was at least five-fold down regulated in all 14 tumors analyzed (Shridhar et al. (2001) Cancer Res. 61:5895-5904).

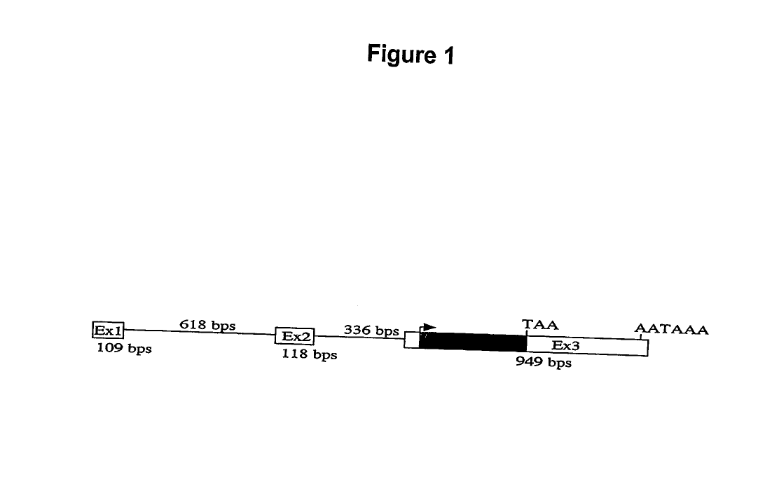

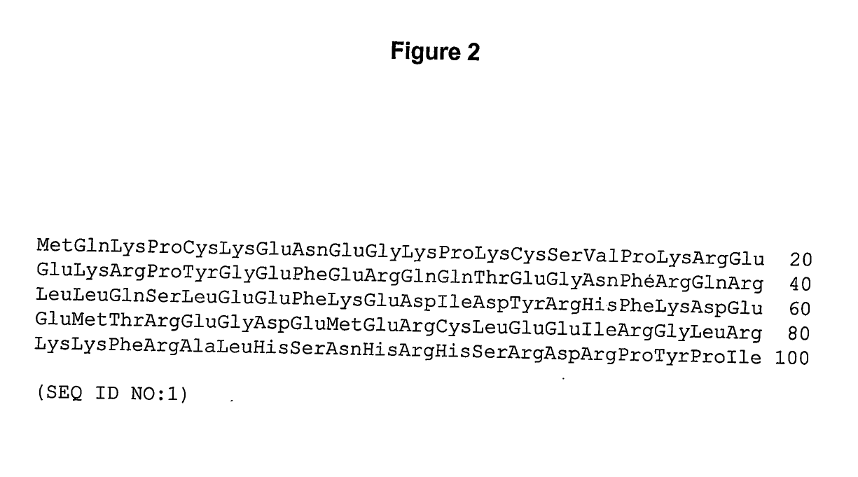

The homologous ESTs were assembled into a contig with the use of Sequencher 4 (Gene Codes Corp) software, and the resulting entire full length cDNA was confirmed by PCR sequencing. The assembled gene is made up of three exons, with the ORF residing entirely within exon 3 (FIG. 1). Exons 1,2, and 3 are 109 bp, 118 bp and 949 bp long respectively. Introns 1 and 2 are 618 bp and 336 bp long respectively. The putative open reading frame encodes a polypeptide that is 100 amino acids in length (FIG. 2; SEQ ID NO:1). The putative initiation codon occurs within a strong Kozak context (Kozak (1996) Mamm. Genome 7:563-574; Kozak (1999) Gene 234:187-208) and is preceded by an in-frame stop codon. The assembled gene mapped to Xq21.1-21.3 based on the Human Genome BLAST server database.

### Example 4

### Similarity Alignment of Bex4 to Other Proteins

To identify potential functions of the assembled gene, a homology search using Blastp was performed (Altschul et al. (1997) Nucleic Acids Res. 25:3389-3402). The search revealed sequence similarity to brain-expressed (Bex) proteins, Bex1-3. The Bex proteins belong to a small family of genes comprised of Bex1 (Faria et al. (1998) Mol. Cell. Endocrinol. 143:155-66), Bex2, and Bex3 (Rapp et al. (1990) DNA Cell. Biol. 9:479-485; and Mukai et al. (2000) J. Biol. Chem. 275:17566-17570). The newly identified gene, which mapped between Bex 2 and 3 on X22.1-22.2, was designated as Bex4.

To verify the transcript size and determine the tissue distribution of Bex4, multiple tissue northern blots and tissue expression arrays were hybridized with a Bex4 probe corresponding to the open reading frame. A single transcript of ˜1.2 kb was detected the northern blot with highest expression in brain, consistent with the expression pattern of other Bex members. Bex4 was expressed in various brain tissues as well as ubiquitously expressed in various other tissues spotted on the array.

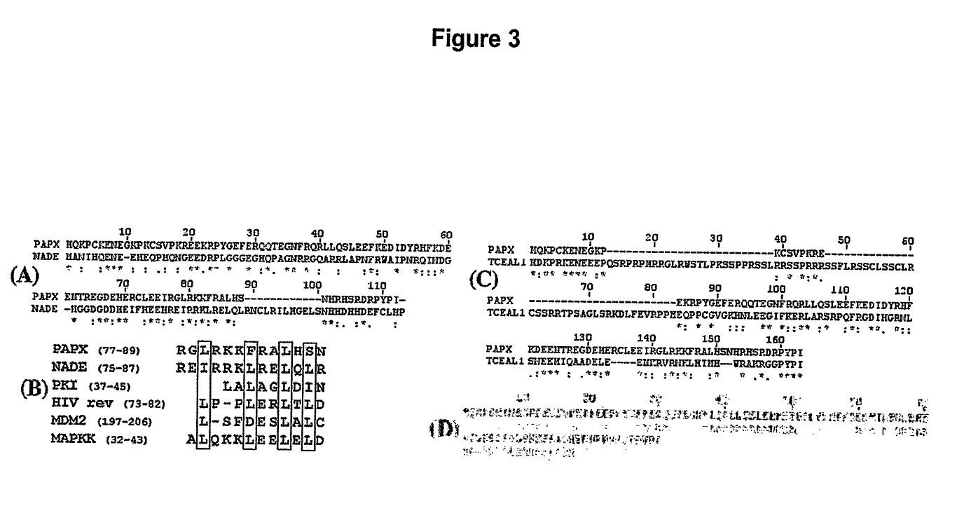

CLUSTALW sequence alignment analyses between Bex4 and two homologous proteins are shown in FIG. 3. The highest identity was observed with the p75NTR-associated death executor (NADE; 37%; FIG. 3A) and with the transcription elongation factor A-like 1 (TCEAL1; 35%; FIG. 3C). Bex4 and NADE share a high degree of homology except between residues 72-112 of NADE, a region essential for NGF-dependent regulation of NADE-induced apoptosis (Mukai et al. (2002) J. Biol. Chem. 277:13973-13982). Alignment of Bex4 with several other polypeptides revealed the presence of a Rev-like NES motif (FIG. 3B). Bex4 and TCEAL1 share homologies in the N— and C-terminal regions, with a long stretch of similarity primarily in the helix-turn-helix motif (FIG. 3D).

### Example 5

### Expression of Bex4 in Cell Lines and Primary Tumors

To validate the results obtained by microarray and SSH analyses, Bex4 expression was tested in seven OvCa cell lines and in 26 primary ovarian tumors by semi-quantitative RT-PCR and northern blot analysis. RT-PCR analysis of ovarian cell lines indicated that Bex4 is not expressed in any of the OvCa cell lines tested, but is expressed in short-term culture OSE and immortalized OSE. Northern blotting produced similar results. RT-PCR analysis of primary ovarian tumors revealed reduced or loss of Bex4 expression in a majority of the tumors tested, as compared to two short-term culture OSEs. Only three of the 24 primary tumors tested showed expression of Bex4 by Northern blot analysis.

Immunoblot analysis also showed down regulation of Bex4 mRNA expression in the SKOV3, OV266, OV207, OV202, and OV207 cell lines. cDNA microarray analysis of 51 ovarian tumors also indicated a consistent down-regulation of the Bex4 transcript. RT-PCR analyses of two other genes, NADE and pp2l homolog, which map close to Bex4, showed only a minimal loss of expression, indicating specific targeting of Bex4 for inactivation in cancer.

Bex4 expression also was examined in cells and cell lines of the following types: cervical (normal keratinocytes, SiHa, CaSki, HT-3, HeLA, SW756, MS751, C-33-A, C-4-, and ME-1 80), hepatocellular carcinomas (Hep3B, HepG2, HUH7, SNU182, SNU387, SNU423, SNU449, SNU475, SKHep1, and PLC5), prostate (benign prostate epithelium [BPH], Du 145, LNCAP, and PC3), breast (normal human mammary epithelial cells [HMEC], MCF-7, MCF-10A, MDA-MB-157, MDA-MB-361, MDA-MB-435, MDA-MB 468, and UACC812), and brain (total brain, D32, D37, M067, T989, U148, U251, and U373), as well as cells from ten primary brain tumor samples. RT-PCR analyses of these cell lines and primary tumors revealed that Bex4 expression was lost or reduced as compared to the level of Bex4 expression in normal cells, indicating that down regulation of Bex4 is common in several epithelial malignancies.

### Example 6

### Mechanism of Bex4 Inactivation

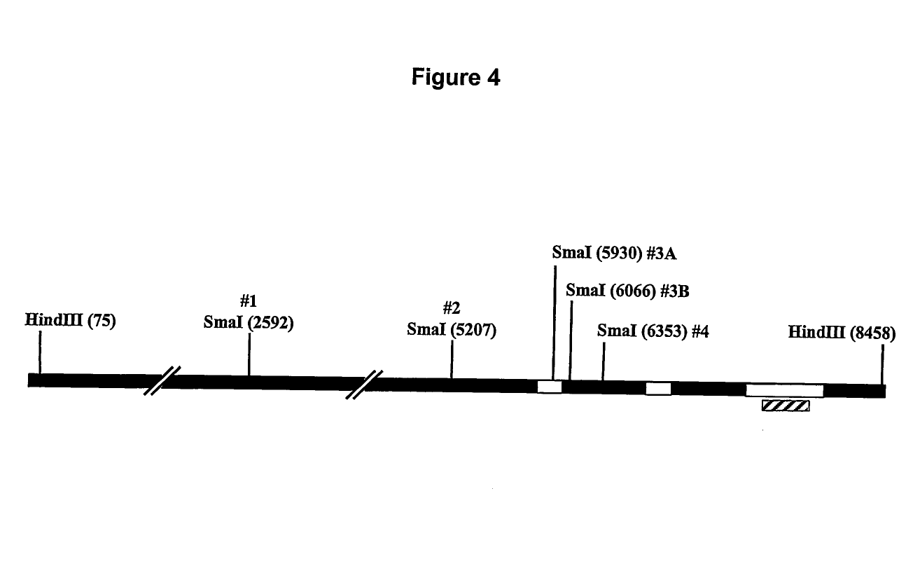

To investigate the mechanism of Bex4 inactivation, 30 pairs of matched normal and primary ovarian tumors were analyzed for LOH using two different microsatellite markers within the Bex4 gene. LOH analysis revealed no deletions in this region of the chromosome. Mutational analysis of all three exons in 96 primary ovarian tumors showed no tumor specific change. However, genomic sequence analysis indicated the presence of putative CpG sites defined by SmaI sites in the promoter, exon 1 and intron 1 (FIG. 4).

Methylation-specific PCR (MS-PCR) studies were conducted. DNA was modified with sodium bisulfite according to Herman et al. ((1996) Proc. Natl. Acad. Sci. USA 93:9821-6982) with the following modifications: 1-1.5 μg of DNA was digested with EcoRI in a 50 μl reaction overnight. The digested DNA was extracted once with phenol-chloroform-isoamyl alcohol (25:24:1) and precipitated with 1/10 volume of 5 M ammonium acetate and 100% ethanol in the presence of 1 μl of 20 mg/ml glycogen (Boehringer Mannheim, Indianapolis, Ind.). The DNA pellet was washed twice with 70% ethanol and the DNA was taken up in 90 μl of 10 mM Tris (pH 7.5) containing 1 mM EDTA (TE buffer). Ten μl of freshly prepared 3 M NaOH was added to each sample and the DNA was denatured at 42° C. for 30 minutes. After addition of 10 μl of distilled water, 1020 μl of 3.0 M sodium bisulfite (pH 5.0) and 60 μl of 10 mM hydroquinone, the samples were incubated in the dark at 55° C. overnight (16-20 hours). Modified DNA was purified using the Wizard purification system (Promega, Madison, Wis.,) according to the manufacturer's instructions, followed by denaturation with 0.3 M NaOH for 15 minutes at 37° C. The DNA was eluted in 50-100 μl of TE and stored at −20° C. in the dark. Primers used in the methylation analysis are listed in Table 1. PCR was performed by the “hot-start” method using Taq gold. An initial denaturation of 10 minutes followed by 40 cycles of amplification at temperatures ranging from 54° C. to 65° C. annealing with primers to amplify methylated sequences and 50° C. annealing to amplify nonmethylated/modified DNA with UF/UR primers. Controls without DNA and positive controls with unmodified DNA were performed for each set of reactions.

TABLE 1
Primers used in methylation analysis

| | | Size | Anneal | SEQ |
|---|---|---|---|---|
| Primer | Position | (bp) | temp | ID |
| MF2: 5′-TGTGATTTATAGTTGCGATTTG-3′ | Promoter | 211 | 54° C. | 40 |
| MR2: 5′-CCAACCACCTATTTCTACTACT-3′ | | | | 41 |
| WTF2: 5′-TGTGACTTACAGTTGCGATGTC-3′ | | | | 42 |
| WTR2: 5′-CCAGCCACCTGTTTCTGCTGCT-3′ | | | | 43 |
| MF3A/B: 5′ ATTAGGAGTTGACGTGAATCG-3′ | Exon 1 | 209 | 54° C. | 44 |
| MR3A/B: 5′-CTATTCCAATCATTAATTCACCC-3′ | Intron1-1 | | | 45 |
| WT3A/BF: 5′ATCAGGAGCTGACGTGAACG-3′ | | | | 46 |
| WT3A/BR: 5′CTATTCCAGTCATTAGATTCACCC-3′ | | | | 47 |
| MF4: 5′-GTATGTGTTGGTGTGTGGAGAAAG-3′ | Intron1-2 | 166 | 65° C. | 48 |
| MR4: 5′-AAACCTAAACCTTACAAAACCGCG-3′ | | | | 49 |
| WTF4: 5′-GTATGTGTTGGTGTGTGGAGAAAG-3′ | | | | 50 |
| WTR4: 5′-AGGCCTGGGCCTTGCAAGGCCCGCG-3′ | | | | 51 |

Methylation analysis of the four SmaI sites was performed in VOSE, OV202, OVCAR5 and SKOV3 cells, and in somatic cell hybrids containing active X (Y162.11C) and inactive X (Y162.5E1T2; Table 2). Bisulfite modified DNAs were amplified, gel purified, and sequenced to determine the methylation status of these sites. Methylated Cs were resistant to bisulfite modification, whereas unmethylated Cs were converted to Ts. Therefore, methylated Cs were read as Gs, and unmethylated Cs (converted to Ts) were read as As in the complementary strand. In VOSE DNA, the SmaI sites showed the presence of both methylated and unmethylated Cs, presumably the result of one allele being methylated and the other being unmethylated. In OV202 cells, which do not express Bex4, only methylated Cs were detected at the SmaI sites.

Similar results were obtained from all cell lines and primary tumors with loss of Bex4 expression. Sites 2 and 4 were methylated on both alleles in the tumor cell lines. In VOSE, however, which express Bex4, these sites showed the presence of both methylated and unmethylated alleles, indicating monoallelic expression of Bex4.

TABLE 2
Methylation status of SmaI sites 2, 3A, 3B, and 4

| Cell line | Promoter site 2 | Exon 1 site 3A | Intron 1 site 3B | Intron 1 site 4 | Bex4 Expression |
|---|---|---|---|---|---|
| VOSE | UNM/M | UNM/UNM | UNM/UNM | UNM/M | + |
| OV167 | M/M | M/M | UNM/UNM | M/M | − |
| OV177 | M/M | M/M | UNM/UNM | M/M | − |
| OV202 | M/M | M/M | UNM/UNM | M/M | − |
| OV207 | M/M | M/M | UNM/UNM | M/M | − |
| OV266 | M/M | M/M | UNM/UNM | M/M | − |
| OVCAR₅ | M/M | UNM/UNM | UNM/UNM | M/M | − |
| SKOV3 | M/M | UNM/UNM | UNM/UNM | M/M | − |
| Inactive X | UNM | M | UNM | M | |
| Active X | UNM | UNM | UNM | UNM | |

The MS-PCR analyses demonstrated monoallelic methylation of sites 2, 3, and 4 in VOSE and in some primary tumors, providing further evidence of monoallelic expression of Bex4. The analyses also indicated biallelic methylation of sites 2, 3 and 4 in some tumors. Examination of the association between biallelic methylation of sites 2, 3 and 4 with loss of Bex4 expression, however, did not reveal any correlation between the methylation of these sites and loss of Bex4 expression. SmaI site 3B was unmethylated on all alleles tested, while SmaI site 3A was unmethylated in OVCAR₅ and SKOV3 and methylated in the rest of the cell lines.

Since Bex4 maps to a region of the X chromosome that contains genes that are only expressed from the active X (Austin (1991) Cancer Genet. Cytogenet. 54:71-76), one of the Bex4 alleles may be subject to X chromosome inactivation (Avner and Heard (2001) Nat. Rev. Genet. 2:59-67) and may be mono-allelically expressed. Methylation of Bex4 on the active X may lead to a complete loss of expression in cell lines not expressing Bex4. Site 4 was differentially methylated in somatic cell hybrids containing either an active or an inactive X chromosome as its only human counterpart; this site was methylated in a hybrid with an inactive X chromosome and unmethylated in a hybrid carrying an active X chromosome. Similarly, site 3A showed a differential methylation pattern in the somatic cell hybrids containing either the active or the inactive X chromosome. Thus, methylation of sites 2, 3, and 4 could represent a developmentally regulated process involved in silencing Bex4 expression during X chromosome inactivation.

Interestingly, Bex4 expression also was not detected in a hybrid cell line containing active X chromosome (Xa) even though 3 of 4 sites are not methylated compared to hybrid cell line containing inactive X chromosome. Sequence analysis of bisulfite-modified DNA showed methylation of site 1 on both Xa and Xi hybrid cell lines. These data suggest that aberrant methylation at site 1 during hybrid cell line propagation could have inactivated Bex4 expression in the Xa cell line.

To determine if hypermethylation is responsible for inactivation of Bex4 expression in OvCa cell lines, OV202 cells were treated with 0, 1, 5, or 10 μM 5-aza-2′-deoxycytidine (a DNA methyltransferase inhibitor) for 48 hours. RNA was extracted, reverse transcribed, and analyzed by PCR using Bex4 specific primers. A dose dependant increase in the transcription of Bex4 was observed in response to 5-aza 2′ deoxycytidine treatment.

To determine if methylation of site 1 controlled Bex4 expression in the Xa cell line, cells were treated with various concentrations of the methyltransferase inhibitor 5-aza-2′ deoxycytidine (5-aza-dC). A dose-dependent increase in Bex4 expression was observed in this cell line following 5-aza-dC treatment. Subsequent analysis of bisulfite-modified DNA from 5-aza-dC treated Xa cells revealed demethylation of site 1 concomitant with induction of Bex4 transcript. These data strongly suggest that methylation at site 1 controls Bex4 expression. In order to rule out the possibility of histone deacetylation as a mechanism of Bex4 transcriptional inactivation, Xa cells were treated with various concentrations of the histone deacetylase inhibitor, trichostatin A. Trichostatin A did not affect the transcription of Bex4 in these cells. Epigenetic inactivation Bex4 in Xa was specific, since two genes flanking Bex4, namely Bex3/NADE and pp21 homolog, were expressed in Xa.

Consistent with methylation as a mechanism of Bex4 inactivation, 5-aza-dC treatment of another cell line (OV207) also induced Bex4 transcription. Since these data indicate that methylation at site 1 controls Bex4 transcription in Xa cell line, experiments were conducted to test whether site 1 methylation in primary tumors and cell lines is associated with loss of Bex4 expression. Methylation at site 1 in these samples was associated with loss of Bex4 expression, as evidenced by semi-quantitative RT-PCR. Consistent with the hypothesis that site 1 methylation controls Bex4 expression, samples with high levels of Bex4 expression (≧1.5 fold over OSE, e.g. tumor 285) showed an absence of methylation on both alleles. Samples with intermediate levels of expression (˜1 fold over OSE, e.g. 97, 220, 461, OSE) showed monoallelic methylation. Several samples with loss of Bex4 expression (e.g., 34, 98, 107, 121, 259, 339) showed biallelic methylation at site 1. However, some samples exhibited loss of Bex4 expression with no correlation to site 1 methylation (e.g. tumors 45, 183, and 235), indicating the involvement of other regulatory factors for inactivating Bex4 expression.

### Example 7

### Cellular Localization of Bex4

To determine the subcellular localization and to characterize the function of Bex4, c-terminal GFP-tagged p16 construct was generated and null ovarian cell line OV202 stably transfected to express the fusion protein. Bex4 localized exclusively to nuclei, compared to widespread distribution of GFP alone. Proliferation index, as indicated by BrdU labeling, also decreased in Bex4 expressing cells. Cell cycle analysis indicated a decrease in S-phase and an increase in G2/M cells, indicating a delay in cell cycle entry and exit. Consistent with the impediment of cell cycle progression, Bex4 expressing cells formed fewer colonies compared to those cells expressing GFP alone. These results established the potential role of Bex4 as a regulator of cell cycle and a candidate tumor suppressor.

Blastp analysis of Bex4 indicated sequence similarities to the transcription elongation factor TCEAL-1 (FIG. 3C) and pp21 homolog. To investigate the role of Bex4 as a transcription regulator, immunolocalization of Bex4 was compared with that of RNA polymerase II. Bex4 co-localized with RNA polymerase II. In addition, Bex4 co-immunoprecipitated with RNA polymerase II, suggesting a possible role as a transcriptional regulator.

To determine whether a decrease in S-phase cells associated with re-expression of Bex4 could be due to transcriptional repression of cyclin D1, a cyclin D1 promoter luciferase construct was co-transfected into HeLa cells either with vector-GFP or a Bex4-GFP fusion construct. These studies showed that cyclin D promoter activity was repressed in cells transfected with Bex4. Western blot and subsequent densitometric analyses of whole cell lysates from three replicates also confirmed the down-regulation of cyclin D1 associated with Bex4 expression. Synchronization of cell cycle with hydroxyurea demonstrated a pronounced delay in reentry into G1 phase, as determined by induction of cyclin D1, in cells transfected with Bex4. In contrast, vector-transfected cells exhibited re-entry and progression through G1-phase within 18 hours after the release from hydroxyurea. Consistent with the role of cyclin D1 in initiating a cascade of events leading to activation of E2F, with subsequent induction of cyclin E (Sherr (1994) Cell 79:551-555), repression of cyclin D by Bex4 attenuated the induction of cyclin E. These results provided a mechanism of cell cycle regulation by a novel transcription regulator, and in part explained a basis for its tumor suppressor activity.

### Example 8

### Forced Expression of Bex4 Induces Apoptosis

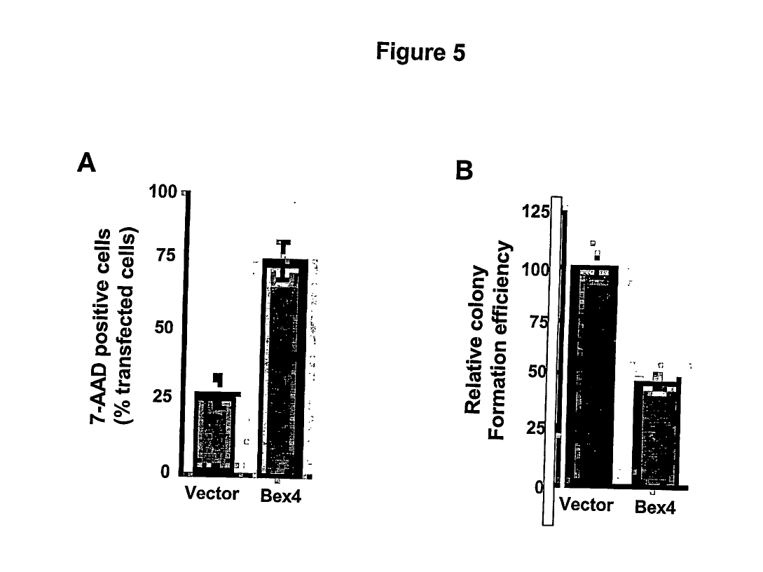

To determine the effect of Bex4 expression in OvCa cells, Bex4 nonexpressing OV202 cells were transiently transfected with a Bex4-GFP fusion construct. Forced expression of Bex4 induced apoptosis and decreased the relative colony formation efficiency of OvCa cells. Phase-contrast microscopy revealed that cells expressing Bex4 were rounded up and showed morphology typical of cells undergoing apoptosis, whereas vector-transfected cells displayed normal morphology. Flow-assisted 7-AAD and Annexin V labeling were used to show that cells expressing Bex4 underwent apoptosis at a markedly higher rate than vector-transfected cells (FIG. 5A). In a colony formation assay, cells transfected with Bex4 formed about 55% fewer colonies than vector-transfected cells (FIG. 5B).

### Example 9

### Bex4 Modulates the Expression of Pro- and Anti-Apoptotic Genes

Two million cells transiently transfected with either vector or Bex4 construct were analyzed for changes in global gene expression using 25K nylon microarrays as previously described (Shridhar et al. (2001) Cancer Res. 61:5895-5904). The top 25 genes that were differentially regulated (at least five-fold or above) by Bex4 are listed in Table 3. Genes implicated in regulating apoptosis or cell survival in various systems are indicated by asterisks. For example, calcineurin, a Ca²⁺ activated protein phosphatase that has been shown to induce apoptosis by dephosphorylating BAD (Wang et al. (1999) Science 284:339-343) was up regulated 13-fold by Bex4. AD7c-NTP, which was up regulated 6.9-fold by Bex4, was shown to promote neuritic sprouting and cell death on overexpression in neuronal cells (Monte et al. (1997) J. Clin. Invest. 100:3093-3104). One of the down-regulated genes (5.65-fold) is mitogen activated protein kinase 2 (MEK2), which is involved in the activation of extracellular signal-related kinase ½ (ERK½) and pro-survival signaling (Zheng and Guan (1993) J. Biol Chem. 268:23933-23939; Zheng and Guan (1993) J. Biol. Chem. 268:11435-11439; Salh et al. (1999) Anticancer Res. 19:731-740; and English and Cobb (2002) Trends Pharmacol. Sci. 23:40-45). Bex4 did not seem to modulate the expression of the typical pro- or anti-apoptotic members of Bc1-2 family or the expression of caspases. However, some of the highly up regulated genes were shown to be involved in either inducing apoptosis or differentiation. For example, interleukin 7R (IL7R), which is ten-fold up-regulated, is involved in inducing differentiation of B-cells (Corcoran et al. (1996) EMBO J 15:1924-1932). To confirm the results generated under transient transfection conditions, stably transfected clonal lines can be evaluated using a different platform (Affymetrix). Bex4 transfected cell lines also can be examined under conditions where Bex4 nuclear foci formation may be enhanced.

TABLE 3
Genes induced or suppressed by Bex4

| | fold up | | fold down |
|---|---|---|---|
| cDNA | Bex4 | cDNA | Bex4 |
| Bex4 | 51.50 | UID = Hs.226124 | 16.75 |
| Calcineurin A* | 13.26 | dbEST = 377234 | 15.39 |
| UID = Hs.299544 | 10.64 | UID = Hs.127557 | 11.91 |
| Interleukin 7 receptor | 10.02 | UID = Hs.180793 | 9.57 |
| UID = Hs.130904 | 9.24 | UID = Hs.221513 | 7.98 |
| UID = Hs.92004 | 9.05 | UID = Hs.298448 | 7.94 |
| SemaF cytoplasmic domain associated | 8.54 | UID = Hs.292833 | 7.93 |
| protein 3 | | | |
| UID = Hs.181054 | 8.52 | Phosphodiesterase 10A | 7.01 |
| Disintegrin reprolysin | 8.34 | UID = Hs.130470 | 6.94 |
| UID = Hs.104705 | 8.13 | UID = Hs.285673 | 6.68 |
| UID = Hs.99546 | 7.90 | UID = Hs.114765 | 5.90 |
| UID = Hs.4791 | 7.86 | UID = Hs.118015 | 5.88 |
| UID = Hs.218260 | 7.75 | UID = Hs.55305 ESTs | 5.88 |
| Prostaglandin E2 receptor | 7.29 | MEK2* | 5.65 |
| UID = Hs.20161 | 7.19 | AK000305 unnamed protein | 5.61 |
| | | product | |
| Mu-crystallin homolog | 7.12 | UID = Hs.65828 | 5.54 |
| AD7c-NTP* | 6.96 | Retinal fascin | 5.41 |
| UID = Hs.107331 | 6.77 | UID = Hs.301584 | 5.34 |
| UID = Hs.163044 | 6.76 | Diphosphomevalonate | 5.32 |
| | | decarboxylase | |
| beta-site amyloid precursor protein | 6.60 | UID = Hs.97661 | 5.25 |
| cleaving enzyme | | | |
| Dermatan/chondroitin sulfate | 6.58 | UID = Hs.288837 | 5.18 |
| 2-sulfotransferase | | | |
| Carboxypeptidase N precursor | 6.40 | UID = Hs.269208 ESTs397620 | 5.12 |
| Mitochondrial 3-oxoacyl-CoA thiolase | 6.38 | Troponin I | 5.07 |
| UID = Hs.292941 | 6.31 | DKFZp434D1812 | 4.93 |
| UID = Hs.117955 | 6.24 | UID = Hs.129990 | 4.78 |

*Genes reported to be involved in apoptosis and/or cell survival.

### Example 10

### The Effect of Bex4 Re-Expression on Malignant Phenotypes of OvCa Cells

Transfection: Over-expression of Bex4 induced apoptosis in SKOV3 cells compared to parental or vector transfected cells. However, high expressing stable Bex4 clones were not selected since all cells over-expressing Bex4 were killed, presumably due to constitutive activation of the apoptotic machinery. To overcome this problem and better define the biological effects of Bex4 re-expression in vitro and in vivo, an OvCa cell line is constructed that expresses Bex4 in a tetracycline inducible manner. This system confers several advantages over transient transfection systems: (1) the ability to maintain stable cell lines, (2) the ability to induce Bex4 expression under controlled conditions, and (3) the ability to clearly delineate the effect of Bex4 expression by turning it on and off in the same clonal cell line. Stable clones of SKOV3 cells are generated that express the pTet-On regulator plasmid (with reverse transactivator which binds TRE only in the presence of doxycycline (Dox) so that the target gene (Bex4) gene stays off until Dox is added) with low background and high Dox dependent induction of luciferase off the pTRE-luciferase plasmid. The Bex4 cDNA encoding the full length ORF is subcloned into pTRE2 plasmid and this cDNA construct is cotransfected with pTK-Hyg plasmid for selecting hygromycin resistant colonies. Four separate clones are isolated that show robust Bex4 induction in DOX. These clones are subsequently used to more carefully to examine the effects of Bex4 on gene expression, growth of SKOV3 cells in soft agar, and ability to form tumors in nude mice.

Tumorigenicity in Nude Mice: A total of 40 mice, with 10 in each group, are injected subcutaneously (s.c.) with 2×10⁷ cells/injection of Bex4 expressing clones #1, #2, #3, or #4 on the left shoulders, and vector expressing clone #1, #2, #3, or #4 on the right shoulders. The transcription of Bex4 is induced in mice in vivo by providing Dox containing drinking water beginning 1 day after implantation. This sample size per condition allows 80% power to determine a 50% change in oncogenic potential that is statistically (and biologically) significant (alpha=0.05). Mice are monitored and growth of subcutaneous tumors is measured in two dimensions daily for 20 weeks, with the tumor volume calculated according to the formula V=a²b/2, where a is the shortest and b the longest diameter. Data are analyzed using repeated measurements ANOVA (analysis of variance) including a growth curve analysis. Animals are observed for up to 20 weeks for measurement of latency time or the failure to develop tumor nodules at the implantation site. At the time of sacrifice, tumors are removed for histological assessment and storage in liquid nitrogen for subsequent studies to ensure that the Bex4 expression is still retained in these samples.

The survival and cell death assays also are repeated with all 4 clonal lines (Bex4 and vector-transfected). DNA laddering experiments and morphological assessment of apoptosis after staining with Hoechst 33258 are used to complement the flow assisted apoptosis assay. Since SKOV3 cells can grow in soft-agar as well as in nude mice in the presence of Matrigel, SKOV3 are used for these experiments.

### Example 11

### Identifying Cellular Proteins that Interact with Bex4

Bex4 shares homology with the RNA binding domains of TCEAL1 and TFIIS. Since these two proteins interact with the RNA Po1 II holocomplex, Bex4 also may influence RNA Po1 II function through a physical interaction. Preliminary studies indicated that Bex4 co-localizes with RNA Po1 II in nuclear foci. Co-immuno-precipitation assays utilizing a monoclonal anti-Bex4 antibody (p2G7) and anti-RNA po1 II are used to investigate an association between these two polypeptides. Bex4 also is highly homologous to the pro-apoptotic protein NADE, suggesting a role for Bex4 in apoptosis. Consistent with both of these results, transcriptional profiling of cells over-expressing Bex4 indicated the up-regulation of several genes that are involved in apoptosis. To better define the function of Bex4, its interaction partners are identified by a yeast two-hybrid screen (e.g., Matchmaker Gal4 Two-Hybrid System 3, Clontech, Palo Alto, Calif.) and/or by microsequencing polypeptides immuno-precipitated with Bex4 monoclonal antibody.

Yeast Two-hybrid Analysis: cDNA encoding full-length Bex4 is subcloned into the bait plasmid pGBKT7 and transformed into the yeast strain AH109. Expression of the resulting GAL4/DBD-Bex4 fusion protein is confirmed by anti-GAL4/DBD and anti-Bex4 by Western blotting. Furthermore, autoactivation of the reporter genes (His3, Ade2 and LacZ) by GAL4/DBD-Bex4 is analyzed, and the optimal concentration of 3-aminotriazole to suppress HIS3 leakiness is determined. Yeast carrying the GAL4-Bex4 bait vector then are mated with the yeast strain Y187 that has been pretransformed with a human fetal brain cDNA fused to the GAL4 activation domain (Clontech). After selection on His⁻/Ade⁻ plates, DNA from clones that are also LacZ positive are extracted, sequenced, and subjected to a BLAST search.

Due to a triple selection, false positives in the screen are minimized. Furthermore, should full-length Bex4 cloned into the bait vector autoactivate even at high 3-aminotriazole concentrations, utilize only fragments of Bex4 are used as a bait. Furthermore, potential interaction partners identified via this technique are validated by co-immunoprecipitation and co-localization assays in mammalian cells. Alternatively, interaction partners are identified by immunoprecipitating Bex4 from cells with anti Bex4 antibody and identifying co-immunoprecipitated proteins by microsequencing.

### Example 12

### Determining the Clinical Significance of CpG Hypermethylation of Bex4

CpG island methylation can be used as a tool to detect cancer cells in serum, urine, bronchoalveolar lavages, and other body fluids. A higher degree of CpG island methylation is associated with early disease and recurrence after chemotherapy in OvCa (Menssen and Hermeking (2002) Proc. Natl. Acad. Sci. USA 99:6274-6279). The presence or absence of Bex4 methylation in peritoneal washings from patients undergoing second look laparotomy or laparoscopy (SLL) may be an indicator of the presence or absence of cancer cells, and may improve the sensitivity of SLL in detecting residual disease in patients previously treated with surgery and/or chemotherapy.

Bex4 methylation is evaluated in peritoneal washings of patients who have no demonstrable evidence of diseases based on histologic or cytologic examination (SLL negative). Patients with no history of cancer who are undergoing surgery for benign conditions also are examined. The detection of hypermethylation is a positive signal that is detected in the context of 1 in 10,000 contaminating normal cells. Bex4 hypermethylation status is determined in 40 cloned DNA molecules per patient sample. If one allows for the possibility of up to 50% contamination by normal cells, some hypermethylated patients might have as few as 75% methylated DNA molecules in their samples. Testing 40 clones per patient and classifying patients as hypermethylated if the percentage of clones is ≧62.5% and nonhypermethylated if the percentage of clones is <62.5% is sufficient to ensure that both the false-positive and false-negative classification rates are less than 5%. Evidence for a difference in relapse rates between Bex4-methylated and Bex4-nonmethylated patients is determined using the log-rank test. These experiments are used to elucidate the role of Bex4 in regulating OvCa growth and tumorigenecity.

### Example 13

### Determination of the Transcriptional Response Induced by Bex4.

Studies are conducted to determine whether Bex4 is a transcription factor or a modulator of transcription, and whether its expression affects global gene expression. cDNA microarray technology is used for determination of changes in global gene expression.

RNA Preparation and Analysis: RNA is prepared from cell lines using the TRIZOL® guanidinium isothiocyanate/phenol extraction procedure (Invitrogen/Life Technologies) followed by further purification using a Qiagen RNA kit as recommended by Affymetrix. RNA concentrations are determined, and the quality of all RNA samples is verified using an Agilent Bioanalyzer before proceeding to the labeling procedures.

RNA Labeling GeneChiP Hybridization, Washing and Scanning: Double-stranded cDNA is synthesized from total RNA. An in vitro transcription reaction is then done to produce biotin-labeled cRNA from cDNA. To permit more rapid hybridization, the cRNA is fragmented in an acetate buffer before hybridization. A test hybridization is performed on a Test3 chip, which contains a small number of genes and permits determination that the background hybridization is proper, correct sites are labeled, and intensity is adequate. Once these quality control checks are performed, hybridization is performed with U133A chips containing 18460 transcripts (14593 unigene clusters, 513 potential additional full-length transcripts, and approximately 3350 housekeeping gene and other internal controls). Subsequently, the GeneChip automated fluidics station is used for washing steps that remove non-specifically bound cRNA target and stain the hybridized probes with streptavidin-phycoerythrin. The hybridized arrays are scanned using a Hewlett Packard GeneArray Scanner and the fluorescence intensity data is analyzed using an Affymetrix Expression Analysis software package (Affymetrix, Santa Clara, Calif.).

The data are analyzed to identify unique candidate genes over- and under-expressed in Bex4 versus Vector transfected cells. Microarray Suite 5.0 (Affymetrix) software is used to scan and quantitatively analyze the data. The per-chip summaries provided by MAS5.0 are used for initial scanning and perusal of the results. The algorithms provided by Affymetrix are reasonable if the data are to be analyzed in a piecemeal fashion rather than with a holistic approach. The primary analysis is based, however, on the model-based methods of Chu et al. ((2002) Mathematical Biosciences 176:35-51). These methods include all of the chips in the experiment in a single analysis with the treatment design itself as a part of the model. Statistically based rules for outliers from a fitted model, applied after fitting all the data, appear superior for detecting and adjusting for artifacts such as smudges, and detection of preparation failures.

Analysis entails comparison between 4 vector- and 4 Bex4-transfected stable cell lines in duplicate. First, two groups of transfectants (Bex4 vs. Vector) are analyzed to determine whether there are differences in global gene expression between the two groups. Pro- and anti-apoptotic genes are grouped together to determine whether there are differences in expression profile between vector- and Bex4-transfected cells. Permutation test methods are used to calibrate the final significance values for both between group comparisons as well as within group tissue comparisons. Genes are prioritized for further study based on statistical significance as well as biological relevance. Cluster analysis methods and visualization software (Eisen et al. (1998) Proc. Natl. Acad. Sci. USA 95:14863-14868 are used to seek groups of genes with similar gene expression profiles across the set of arrays. Each experiment is performed at least twice (starting with cells and ending with hybridization data).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A vector comprising an isolated nucleic acid encoding a polypeptide having the amino acid sequence set forth in SEQ ID NO:1 or a fragment thereof.

2. A vector comprising an isolated nucleic acid encoding a Bex4 polypeptide, wherein the amino acid sequence of said Bex4 polypeptide comprises a variant relative to the amino acid sequence set forth in SEQ ID NO:1.

3. A method for killing a tumor cell, said method comprising administering to said tumor cell a nucleic acid that encodes a Bex4 polypeptide.

4. The method of claim 3, wherein said Bex4 polypeptide has the amino acid sequence set forth in SEQ ID NO:1 or a fragment thereof.

5. The method of claim 3, wherein the amino acid sequence of said Bex4 polypeptide comprises a variant relative to the amino acid sequence set forth in SEQ ID NO:1.

6. The method of claim 3, wherein a vector comprising said nucleic acid is administered to said tumor cell.

7. The method of claim 3, wherein said tumor cell is selected from the group consisting of an ovarian tumor cell, a cervical tumor cell, a brain tumor cell, a breast tumor cell, a prostate tumor cell, and a hepatic tumor cell.

8. A method for killing a tumor cell, said method comprising administering to said tumor cell a purified Bex4 polypeptide.

9. The method of claim 8, wherein said Bex4 polypeptide has the amino acid sequence set forth in SEQ ID NO:1 or a fragment thereof.

10. The method of claim 8, wherein the amino acid sequence of said Bex4 polypeptide comprises a variant relative to the amino acid sequence set forth in SEQ ID NO:1.

11. The method of claim 8, wherein said tumor cell is selected from the group consisting of an ovarian tumor cell, a cervical tumor cell, a brain tumor cell, a breast tumor cell, a prostate tumor cell, and a hepatic tumor cell.

12. A method for determining the predisposition of an individual to develop cancer, said method comprising measuring the level of Bex4 polypeptide in a biological sample from said individual.

13. The method of claim 12, wherein said individual is predisposed to develop cancer if said level of Bex4 polypeptide in said cells is lower than the level of Bex4 polypeptide in a biological sample from a normal individual.

14. The method of claim 12, wherein said cancer is selected from the group consisting of ovarian cancer, breast cancer, prostate cancer, cervical cancer, brain cancer, and liver cancer.

15. A method for detecting cancer recurrence in an individual diagnosed with and treated for cancer, said method comprising measuring the level of Bex4 methylation in a biological sample from said individual.

16. The method of claim 15, wherein the presence of hypermethylation indicates cancer recurrence, and wherein the absence of hypermethylation indicates that cancer has not recurred.

17. The method of claim 15, wherein said cancer is selected from the group consisting of ovarian cancer, breast cancer, prostate cancer, cervical cancer, brain cancer, and liver cancer.
